(19) United States
(12) Patent Application Publication
(10) Pub. No.: US 2017/0215710 A1
(43) Pub. Date: Aug. 3, 2017

(54) ENDOSCOPE SYSTEM

(71) Applicant: OLYMPUS CORPORATION, Tokyo (JP)

(72) Inventors: Yasuhito KURA, Tokyo (JP); Kazuki HONDA, Tokyo (JP); Kento HASHIMOTO, Tokyo (JP)

(73) Assignee: OLYMPUS CORPORATION, Tokyo (JP)

(21) Appl. No.: 15/492,108

(22) Filed: Apr. 20, 2017

Related U.S. Application Data
(63) Continuation of application No. PCT/JP2015/079703, filed on Oct. 21, 2015.

(30) Foreign Application Priority Data
Nov. 25, 2014 (JP) 2014-238022

(51) Int. Cl. A61B 1/00; A61B 1/05; H04N 5/225; G02B 23/24; H04N 5/235; A61B 1/045; A61B 1/005

(52) U.S. Cl. CPC A61B 1/00177; A61B 1/00009; A61B 1/045; A61B 1/05; A61B 1/00096; A61B 1/0051; G02B 23/2484; G02B 23/243; H04N 5/2351; H04N 5/2256; H04N 2005/2255

(57) ABSTRACT

An endoscope system includes an insertion portion, an objective optical system provided on the insertion portion and configured to form an object image, an image pickup unit configured to pick up the object image, a bending portion configured to cause a distal end portion of the insertion portion to bend, an image processing portion configured to perform cutout processing on the image picked up by the image pickup unit, such that an area of field of view expands in a bending direction of the distal end of the insertion portion by the bending portion, and an exposure control portion configured to perform exposure control based on a brightness of the cutout image.

### CROSS REFERENCE TO RELATED APPLICATION

This application is a continuation application of PCT/JP2015/079703 filed on Oct. 21, 2015 and claims benefit of Japanese Application No. 2014-238022 filed in Japan on Nov. 25, 2014, the entire contents of which are incorporated herein by this reference.

### BACKGROUND OF THE INVENTION

1. Field of the Invention

The present invention relates to an endoscope system, and more particularly, to an endoscope system that obtains an object image from at least two directions.

2. Description of the Related Art

Conventionally, endoscopes have been widely used in a medical field and an industrial field. An endoscope is provided with illuminating means and observing means on a distal end side of an insertion portion, and is able to be inserted into a subject to observe, examine, and treat and the like the inside the subject, for example.

A bending portion is provided on a proximal end side of the distal end portion of the insertion portion. When a user of the endoscope, who is a technician or an examiner, performs an examination or the like, the user is able to perform the examination by displaying an endoscope image on a monitor while bending the bending portion.

Also, in recent years, an endoscope having a wide angle of field of view capable of observing in two or more directions has been proposed. For example, an endoscope capable of observing a side view, in which the observation field of view is to a side face side of the insertion portion, in addition to a front view, in which the observation field of view is front side of the insertion portion, has been proposed, as described in Japanese Patent Application Laid-Open Publication No. 2013-544617. Using such an endoscope enables the user to simultaneously observe two directions, i.e., to the front and to the side, so a wider area can be observed.

### SUMMARY OF THE INVENTION

An endoscope system according to one aspect of the present invention has an insertion portion configured to be inserted into a subject, an objective optical system provided on a distal end of the insertion portion and configured to form an optical image of an object inside the subject, an image pickup portion configured to pick up the optical image, a bending portion configured to cause the distal end of the insertion portion to bend, a bending direction detection portion configured to detect a bending direction of the distal end of the insertion portion by the bending portion, an image processing portion configured to perform cutout processing on the image picked up by the image pickup portion, such that an area of field of view expands in the bending direction detected by the bending direction detection portion, and an exposure control portion configured to perform exposure control based on a brightness of the image cut out by the image processing portion.

### BRIEF DESCRIPTION OF THE DRAWINGS

FIG. 1 is a configuration diagram showing the configuration of an endoscope system according to a first embodiment of the present invention;

FIG. 2 is a sectional view of a distal end portion 5a of an insertion portion 5 according to the first embodiment of the present invention;

FIG. 3 is a view illustrating an example of a display screen of an endoscope image displayed on a display portion 4, and an object image region of an image pickup device 14a of an image pickup unit 14, according to the first embodiment of the present invention;

FIG. 4 is a flowchart showing an example of a flow of image processing according to a bending operation in a control portion 21, according to the first embodiment of the present invention;

FIG. 5 is a view illustrating an area where a region of an image to be displayed on the display portion 4 is cut out from an object image taken on an image pickup surface of the image pickup device 14a, according to the first embodiment of the present invention;

FIG. 6 is a view of a cutout area CA and a display image 41 of the display portion 4, according to a first modification of the first embodiment of the present invention;

FIG. 7 is a view of a cutout area CA and a display image 41 of the display portion 4, according to a second modification of the first embodiment of the present invention;

FIG. 8 is a view of a plurality of division regions DA for an exposure determination in a cutout area CA, according to a third modification of the first embodiment of the present invention;

FIG. 9 is a view of a cutout area CA and a display image 41 of the display portion 4, according to a fourth modification of the first embodiment of the present invention.

FIG. 10 is a configuration diagram showing a configuration of an endoscope system according to a second embodiment of the present invention;

FIG. 11 is a view showing an example of a display screen of an endoscope image displayed on a display portion 4A, according to the second embodiment of the present invention;

FIG. 12 is a view illustrating an example of the display screen of the endoscope image displayed on the display portion 4A, and an object image region of three image pickup units 11a, 11b, 11c, according to the second embodiment of the present invention;

FIG. 13 is a view illustrating a change in a cutout area when cutting out a region of an image to be displayed on the display portion 4A from an object image taken on each image pickup surface of the three image pickup units 11a, 11b, 11c, according to the second embodiment of the present invention;

FIG. 14 is a view showing a state in which a cutout area of each region ORa, ORb, ORc is made to move in a bending direction according to a bending operation amount, according to the second embodiment of the present invention;

FIG. 15 is a view illustrating a display state of the display portion 4A when bending is performed toward a right side, according to a fifth modification of the second embodiment of the present invention;

FIG. 16 is a view illustrating another example of a display state of the display portion 4A when bending is performed toward the right side, according to the fifth modification of the second embodiment of the present invention; and

FIG. 17 is a perspective view of a distal end portion 5a of the insertion portion 5 to which a unit for side observation is attached, according to a sixth modification of the second embodiment of the present invention.

### DETAILED DESCRIPTION OF THE PREFERRED EMBODIMENTS

Hereinafter, embodiments of the present invention will be described with reference to the drawings.

### First Embodiment

### (Configuration)

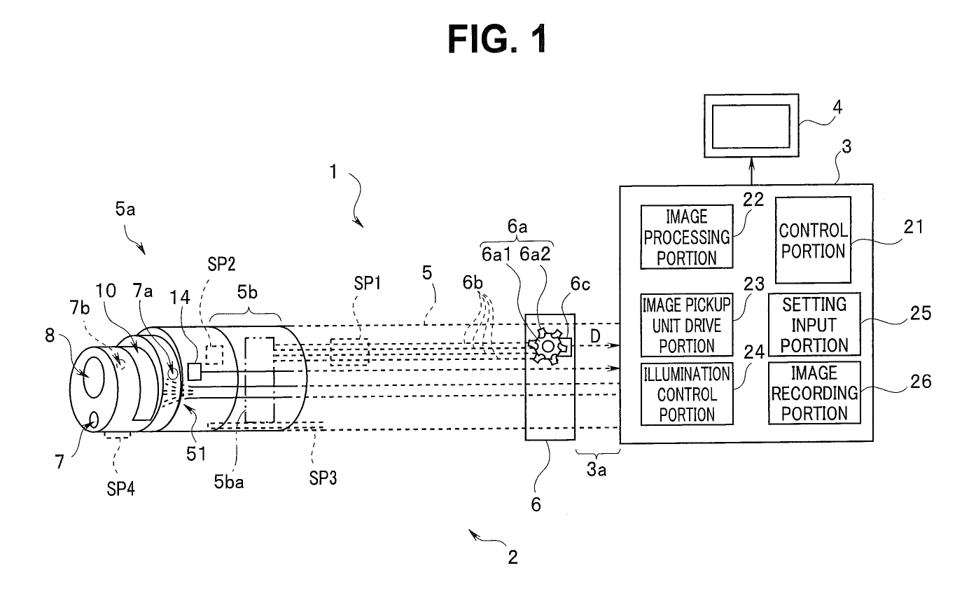

FIG. 1 is a configuration diagram showing a configuration of an endoscope system according to the embodiment. The endoscope system 1 includes an endoscope 2, a processor 3, and a display portion 4.

The endoscope 2 includes an insertion portion 5 that is flexible and is configured to be inserted into a subject, and an operation portion 6 that is connected to a proximal end of the insertion portion 5. The operation portion 6 is connected to the processor 3 by a universal cord 3a. An illumination window 7 and an observation window 8 for a front view, and two illumination windows 7a, 7b and an observation window 10 for a side view, are provided on a distal end portion 5a of the insertion portion 5. The observation window 10, which is an image obtaining portion, is disposed farther toward the proximal end side of the insertion portion 5 than the observation window 8, which is an image obtaining portion.

Also, a light guide 51 formed by an optic fiber bundle is used for illumination. Illumination light for the three illumination windows 7, 7a, 7b enters the proximal end portion of the light guide 51. The distal end portion of the light guide 51 is divided into three, and is disposed behind the three illumination windows 7, 7a, 7b.

Also, a bending portion 5b is provided on the proximal end side of the distal end portion 5a of the flexible insertion portion 5. The bending portion 5b has a bending mechanism 5ba, such as a mechanism in which a plurality of bending pieces are provided in a continuous fashion that enables bending in the up and down and left and right directions, and a so-called swing mechanism that enables pivoting about a predetermined axis such that an optical axis direction of an image obtaining portion can be changed. That is, the bending portion 5b configures a swing portion that changes the direction in which the distal end portion of the insertion portion 5 faces.

A bending knob 6a as a bending operation portion is provided on the operation portion 6. A plurality of bending wires 6b that are connected to the bending mechanism 5ba are pulled taut or are loosened by operating the bending knob 6a, thus enabling the user to bend the bending portion 5b in a desired direction. That is, the bending knob 6a is an operation member that is able to operate so as to change an angle between the direction in which the distal end portion of the insertion portion 5 faces, and a predetermined direction, here, a longitudinal axis direction.

The bending portion 5b is able to bend in the up and down and left and right directions, for example. The bending knob 6a has two knobs 6a1, 6a2, i.e., an up-down direction knob and a left-right direction knob, and four of the bending wires 6b are connected to a distal end bending piece of the bending mechanism 5ba and the bending knob 6a.

Note that the bending portion 5b may be bendable in only two directions, e.g., in only the up and down directions.

A potentiometer 6c that detects a bending operation amount with respect to the insertion portion 5 is provided on the bending knob 6a. The potentiometer 6c as a bending operation amount detection device has two potentiometers that output voltage signals in accordance with pivot amounts of respective shafts of the two knobs 6a1, 6a2 that are the up-down direction knob and the left-right direction knob. When the bending knob 6a is operated by the user, the voltage signal according to the operation amount of each knob 6a1, 6a2 is supplied to a control portion 21 of the processor 3 as a detection signal D.

Note that, here, the potentiometer 6c is used as the bending operation amount detection device, but the bending operation amount may also be detected according to another method, as shown by a dotted line in FIG. 1. For example, a tension meter SP1 may be provided on each bending wire in the up and down and left and right directions, and the bending direction and the bending operation amount may be detected by tension applied to each of the bending wires 6b. Alternatively, an acceleration sensor (or a gyro sensor) SP2 may be provided on a distal end rigid portion of the distal end portion 5a, and the bending direction and the bending operation amount may be detected based on the detected acceleration. Alternatively, a plurality of rod-shaped bending sensors SP3 may be provided in the axial direction of the insertion portion 5, on the bending portion 5b, and the bending direction and the bending operation amount may be detected based on the bending amount detected by the respective bending sensors SP3. Alternatively, a plurality of distance sensors SP4 that measure a distance between a distal end of a flexible tube portion and an outer peripheral portion of the distal end portion 5a in the up and down and left and right directions using a laser, infrared, or ultrasound or the like may be provided, and the bending direction and the bending operation amount may be detected based on the respective detected distances.

Moreover, a pressure sensor (not shown) that detects abutment against an interior wall or the like in the subject when bending in the up and down and left and right directions may be provided on an outer peripheral portion of the distal end portion 5a, and the bending direction and the bending operation amount may be detected based on the contact pressure between the pressure sensor and the internal wall or the like in the subject.

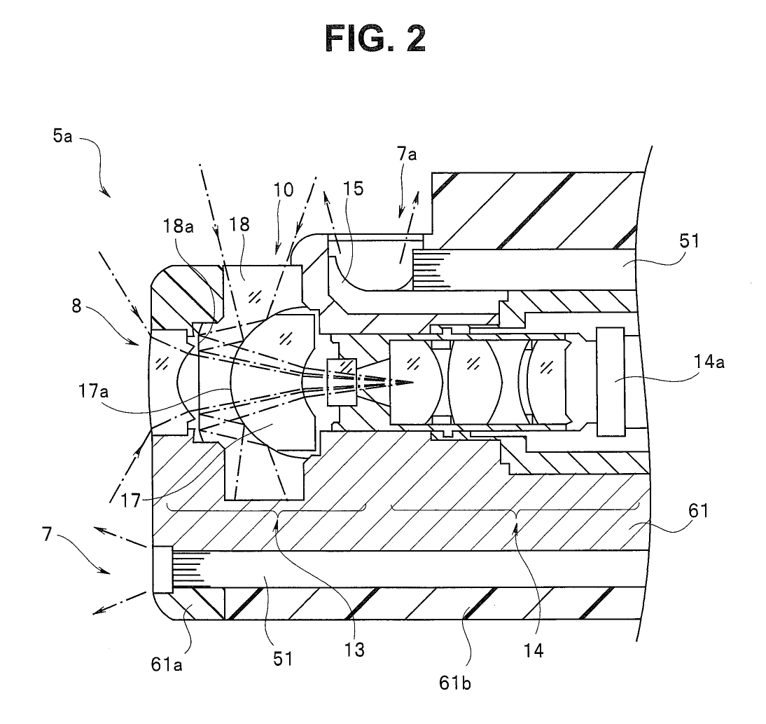

FIG. 2 is a sectional view of the distal end portion 5a of the insertion portion 5. Note that FIG. 2 shows a cross-section in which the distal end portion 5a is cut such that cross-sections of the illumination window 7a for the side view, the illumination window 7 for front illumination, and the observation window 8 for the front view can be seen.

A distal end surface of a portion of the light guide 51 is arranged behind the illumination window 7. The observation window 8 is provided on a distal end surface of a distal end rigid member 61. An objective optical system 13 is arranged behind the observation window 8.

An image pickup unit 14 is arranged behind the objective optical system 13. Note that a cover 61a is attached to a distal end portion of the distal end rigid member 61. Also, the insertion portion 5 is covered by an outer covering 61b.

Accordingly, front illumination light is emitted from the illumination window 7, and reflected light from an object that is the observed portion in the subject enters the observation window 8.

The two illumination windows 7a, 7b are arranged on an outer peripheral surface of the distal end rigid member 61 such that illumination light beams are emitted in directions opposite to each other, and a distal end surface of a portion of the light guide 51 is arranged, via a mirror 15 having a reflective surface that is a curved surface, behind the respective illumination windows 7a, 7b.

Accordingly, the illumination window 7 and the plurality of illumination windows 7a, 7b configure an illumination light emitting portion that emits first illumination light in a region that includes the front direction as a first region, and second illumination light in a region that includes the side direction as a second region that differs from the first region inside the subject.

The observation window 10 is arranged on a side surface of the distal end rigid member 61, and the objective optical system 13 is arranged behind the observation window 10. The objective optical system 13 is configured to direct the reflected light from the front direction that has passed through the observation window 8, and the reflected light from the side direction that has passed through the observation window 10, toward the image pickup unit 14. In FIG. 2, the objective optical system 13 has two optical members 17 and 18. The optical member 17 is a lens having a convex surface 17a, and the optical member 18 has a reflective surface 18a that reflects light from the convex surface 17a of the optical member 17 toward the image pickup unit 14 via the optical member 17.

That is, the observation window 8 configures a first image obtaining portion that is provided on the insertion portion 5, and obtains a first image (first object image) from the first region that is the region that includes the front direction, and the observation window 10 configures a second image obtaining portion that is provided on the insertion portion 5, and obtains a second image (second object image) from the second region that is the region that includes the side direction and that differs from the first region.

More specifically, an image from the first region that includes the front direction is an object image in a first direction that includes the direction in front of the insertion portion 5, which is substantially parallel to a longitudinal direction of the insertion portion 5, and an image from the second region that includes the side direction is an object image in a second direction that includes a direction to the side of the insertion portion 5, which is substantially orthogonal to the longitudinal direction of the insertion portion 5. The observation window 8 is a front image obtaining portion that obtains an object image of a first region that includes a direction in front of the insertion portion 5, and the observation window 10 is a side image obtaining portion that obtains an object image of a second region that includes a direction to the side of the insertion portion 5.

The second region being different from the first region indicates that optical axes in the regions are pointing in different directions. Portions of the first object image and the second object image may or may not overlap, and further, portions of the illumination area of the first illumination light and the illuminated area of the second illumination light may or may not overlap.

Also, the observation window 8 that is the image obtaining portion is disposed, facing in the direction in which the insertion portion 5 is inserted, on the distal end portion 5a of the insertion portion 5, and the observation window 10 that is the image obtaining portion is disposed, facing an outer radial direction of the insertion portion 5, on a side surface portion of the insertion portion 5. The image pickup unit 14 that is the image pickup portion is disposed, and electrically connected to the processor 3, so as to photoelectrically convert the object image from the observation window 8 and the object image from the observation window 10 on the same image pickup surface.

That is, the observation window 8 is disposed, so as to obtain the first object image from the direction in which the insertion portion 5 is inserted, on the distal end portion in the longitudinal direction of the insertion portion 5, and the observation window 10 is disposed in the circumferential direction of the insertion portion 5, so as to obtain the second object image from the second direction. Also, the image pickup unit 14 that is electrically connected to the processor 3 photoelectrically converts the first object image and the second object image on one image pickup surface, and supplies an image pickup signal to the processor 3.

Accordingly, the front illumination light is emitted from the illumination window 7, and the reflected light from the object enters the image pickup unit 14 through the observation window 8, and the side illumination light is emitted from the two illumination windows 7a, 7b, and the reflected light from the object enters the image pickup unit 14 through the observation window 10. An image pickup device 14a of the image pickup unit 14 photoelectrically converts the optical image of the object and outputs an image pickup signal to the processor 3.

Returning to FIG. 1, the image pickup signal from the image pickup unit 14 is supplied to the processor 3 that is an image generating portion, and an endoscope image is generated. The processor 3 outputs the endoscope image that is an observation image to the display portion 4.

The processor 3 includes a control portion 21, an image processing portion 22, an image pickup unit drive portion 23, an illumination control portion 24, a setting input portion 25, and an image recording portion 26.

The control portion 21 includes a central processing unit (CPU), and ROM, RAM and the like, and controls an overall endoscope apparatus. An image processing program executed at the time of a bending operation, described later, is stored in the ROM.

The image processing portion 22 generates a display signal from a signal of the endoscope image to be displayed on the display portion 4 from an image taken based on the image pickup signal from the image pickup unit 14, and outputs the generated display signal to the display portion 4, under the control of the control portion 21.

In particular, the image processing portion 22 generates the image taken in the image pickup unit 14, cuts out a front image and a side image, changes the cutout area, enlarges or reduces the cutout image and the like, under the control of the control portion 21.

The image pickup unit drive portion 23 is connected to the image pickup unit 14 by a signal wire, not shown. The image pickup unit drive portion 23 drives the image pickup unit 14 under the control of the control portion 21. The driven image pickup unit 14 generates the image pickup signal and provides the signal to the image processing portion 22.

The illumination control portion 24 is a light source apparatus that houses a lamp, lets illumination light into the proximal end of the light guide 51, and controls the on/off and light amount of that illumination light under the control of the control portion 21. The control portion 21 performs exposure control of the endoscope image by controlling the illumination control portion 24.

The setting input portion 25 is formed by a keyboard or various operation buttons or the like, and is an input apparatus that the user uses to input operation commands and settings and the like related to the various functions of the endoscope system 1. The control portion 21 sets and inputs setting information and operation command information inputted in the setting input portion 25, into respective processing portions such as the image processing portion 22.

The image recording portion 26 is a recording portion that records the endoscope image generated in the image processing portion 22 under the control of the control portion 21, and includes non-volatile memory such as a hard disk drive or the like.

The image recorded in the image recording portion 26 is able to be selected by setting. The user is able to set a recorded target image recorded in the image recording portion 26, in the setting input portion 25. More specifically, the user is able to set the recorded target image such that only an endoscope image in which the cutout area has been changed according to a bending operation such as that described later, and which is displayed on the display portion 4, is recorded, only an endoscope image before the cutout area is changed according to a bending operation is recorded, or both the endoscope image in which the cutout area has been changed according to a bending operation, and which is displayed on the display portion 4, and the endoscope image before the cutout area is changed according to a bending operation, are recorded.

Note that when both the endoscope image in which the cutout area has been changed according to a bending operation, and which is displayed on the display portion 4, and the endoscope image before the cutout area is changed according to a bending operation, are recorded, both are recorded linked based on time information, so that when viewing the images after the examination, both can be reviewed linked together.

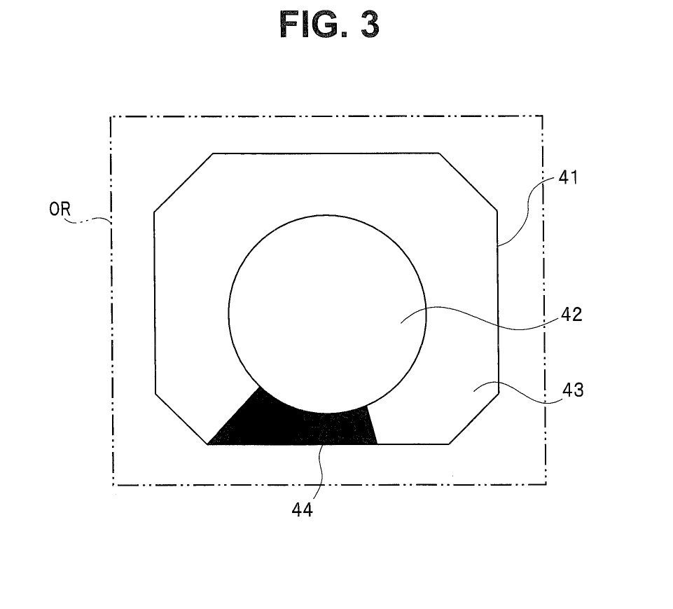

FIG. 3 is a view illustrating an example of a display screen of an endoscope image displayed on the display portion 4, and an object image region of the image pickup device 14a of the image pickup unit 14.

A display image 41 that is the endoscope image displayed on the screen of the display portion 4 is an image with a generally rectangular shape, and has two regions 42 and 43. The region 42 that is circular in the center portion is a region displaying a front view image, and the region 43 that is C-shaped around the region 42 in the center portion is a region displaying a side view image. FIG. 3 shows a state in which both the front view image and the side view image are displayed. The image processing portion 22 outputs an image signal of the front view image and an image signal of the side view image, such that the side view image is displayed around the front view image on the display portion 4.

That is, the front view image is displayed on the screen of the display portion 4 in a generally circular shape, and the side view image is displayed on the screen in a generally annular shape surrounding at least a portion around the front view image. Accordingly, a wide angle endoscope image is displayed on the display portion 4.

The endoscope image shown in FIG. 3 is generated from a taken image obtained by the image pickup device 14a. The front view image and the side view image are generated by being cut out from the object image taken on the image pickup surface of the image pickup device 14a. In FIG. 3, a region OR indicated by the dotted line represents the area of the object image formed on the image pickup surface of the image pickup device 14a.

The display image 41 is generated by photoelectrically converting the object image projected on the image pickup surface of the image pickup device 14a with the optical system shown in FIG. 2, and cutting out a front view image region in the center corresponding to the region 42, and a side view image region corresponding to the region 43, except for a region 44 that is colored black as a masked region, from the region OR of the object image formed on the image pickup surface of the image pickup device 14a, and combining the regions together. The region of the display image 41 in FIG. 3 is the cutout area from the region OR.

The user is able to make the endoscope system 1 execute a desired function by giving the processor 3 an execution command for the desired function. While causing the function to be executed, the user inserts the insertion portion 5 into the subject, and the inside of the subject can be observed and the like while making the bending portion 5b bend.

The user is able to make various settings in the endoscope system 1, including also function settings such as the function settings described later, from the setting input portion 25.

Various settings related to the embodiment include various settings such as whether to change the cutout area of an image according to a bending operation of the bending portion 5, whether to not display a halation region, whether to correct the cutout area when halation occurs, and whether to make a halation region the proper exposure. The details of the settings are recorded in memory or the like in the control portion 21, and when a setting is changed, the details of the setting is changed to the changed details.

The user is able to make the desired settings and change of the settings in the setting input portion 25 before or during an endoscopic examination.

### (Operation)

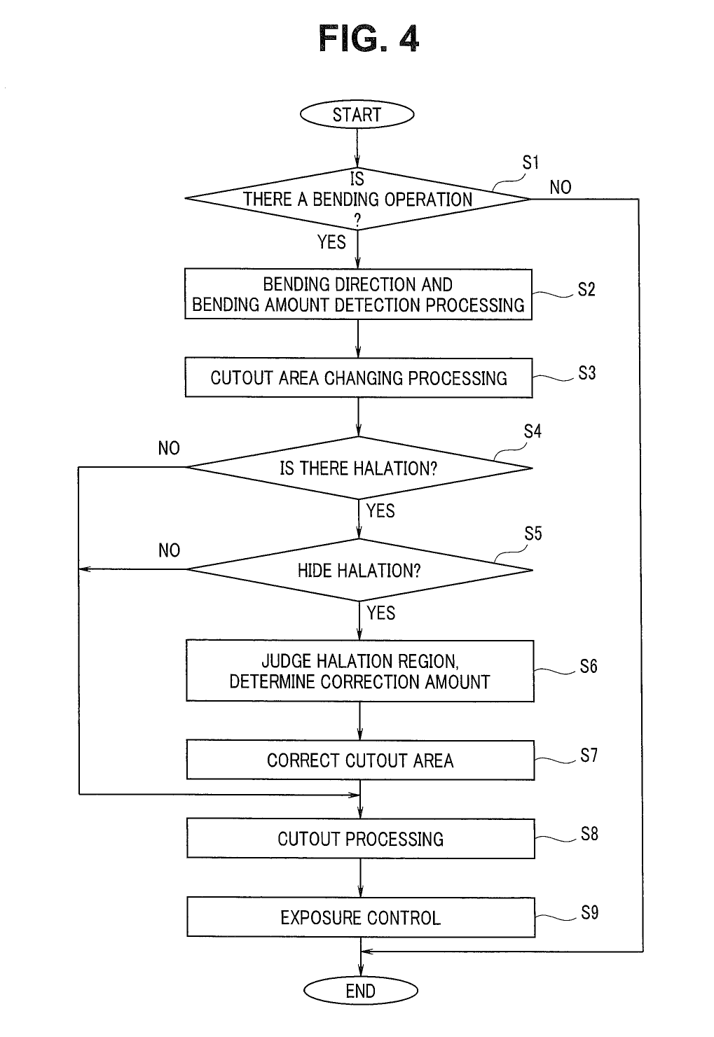

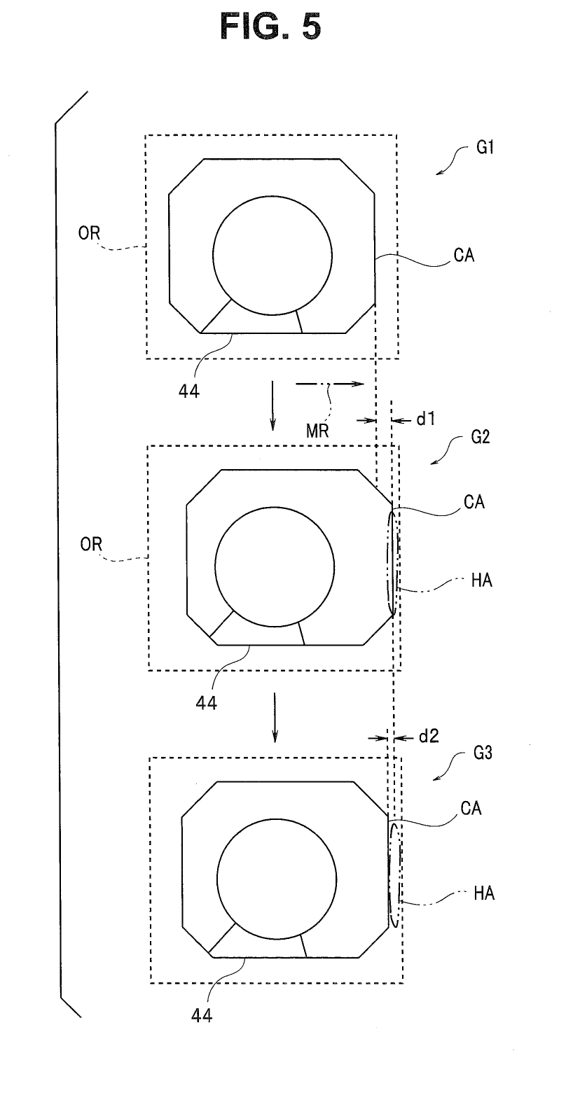

Next, the operation of the endoscope system 1 will be described. FIG. 4 is a flowchart showing an example of a flow of image processing according to a bending operation in the control portion 21. FIG. 5 is a view illustrating an area where a region of an image displayed on the display portion 4 is cut out from the object image taken on the image pickup surface of the image pickup device 14a.

G1 in FIG. 5 is a view showing the cutout area CA of the image displayed on the display portion 4 from the object image taken on the image pickup surface of the image pickup device 14a, when a bending operation is not performed.

The cutout area CA following the shape of the display image 41 has a generally rectangular shape, and has two regions 42 and 43. The region 42 that is circular in the center portion is a region displaying the front view image, and the region 43 that is C-shaped around the region 42 in the center portion is a region displaying the side view image.

The region OR indicated by the dotted line in FIG. 5 shows the area of an object image formed on the image pickup surface of the image pickup device 14a. The display image 41 is generated by photoelectrically converting the object image projected on the image pickup surface of the image pickup device 14a with the optical system shown in FIG. 2, and cutting out a front view image region in the center corresponding to the region 42, and a side view image region corresponding to the region 43, except for the region 44 that is colored black as a masked region, from the region OR, as the cutout area CA, and combining the regions together.

The image processing portion 22 cuts out a predetermined region such as the region shown in G1 in FIG. 5 from the object image taken on the image pickup surface of the image pickup device 14a, as the cutout area CA, and generates an image to be displayed on the display portion 4, when a bending operation is not performed.

The user pushes the insertion portion 5 into a lumen of the subject and observes the interior wall inside the lumen, while inserting the insertion portion 5 into the lumen and making the bending portion 5b bend. For example, during a colon examination, the insertion portion 5 is inserted up to predetermined position inside the large intestine, and the observation is performed while pulling out the insertion portion 5 from the position.

The control portion 21 judges whether a bending operation was performed, based on a detection signal D from the potentiometer 6c of the bending knob 6a (S1). The process of S1 configures a change detection portion that detects a change in the direction in which the distal end portion of the insertion portion 5 faces with respect to a predetermined direction, here, the longitudinal axis direction of the insertion portion 5.

If no bending operation was performed (S1: NO), nothing occurs in the process.

If it is determined that a bending operation was performed (S1: YES), the control portion 21 judges the bending direction and the bending operation amount from the detection signal D, and executes bending direction and bending amount detection processing that detects the bending direction and bending angle of the distal end portion 5a, based on the judged bending direction and bending operation amount (S2). The process of S2 configures a change amount detection portion that detects the direction in which the distal end portion of the insertion portion 5 faces and a change amount in the direction, with respect to a predetermined direction, here, the longitudinal axis direction of the insertion portion 5.

That is, in the process in S2, the angle formed between the direction in which the distal end portion of the insertion portion 5 faces and the longitudinal axis direction of the insertion portion 5, is detected as the amount of change in the direction in which the distal end portion of the insertion portion 5 faces, by an operation for changing the bending angle of the bending portion 5b, with respect to the bending knob 6a that is an operation member.

The control portion 21 executes a cutout area change process that changes the cutout area from the object image taken on the image pickup surface of the image pickup device 14a, based on the direction in which the distal end portion of the insertion portion 5 faces and the change amount in the direction (S3).

That is, the image processing portion 22 generates an image signal that includes the front view image and at least one side view image, and when a change in the direction in which the distal end portion of the insertion portion 5 faces is detected in the process of S1, i.e., the change detection portion, the image processing portion 22 changes the display region included in the image signal of the side view image according to the amount of the change. In particular, the image signal of the side view image is changed so as to include an image of a region not displayed on the display portion 4, in the direction of change in the direction in which the distal end portion of the insertion portion 5 faces.

As shown in FIG. 3, the image pickup device 14a of the image pickup unit 14 is an image pickup apparatus that has an image pickup surface that picks up a wider region than the display image 41 displayed on the display portion 4, which includes the front view image and the side view image. Also, when a change in the direction in which the distal end portion of the insertion portion 5 faces is detected, the image processing portion 22 changes the image signal of the side view image by cutting out a region to include an image in the direction of the change, from the wider region than the display image 41 that was picked up on the image pickup surface.

Note that, here, the cutout area is changed based on the direction in which the distal end portion of the insertion portion 5 faces and the change amount in the direction, but the cutout area may also be changed based on the bending direction and bending operation amount with respect to the bending knob 6a by the user.

Also, the control portion 21 judges whether halation is included in the changed cutout area (S4). Whether halation is included is determined according to, for example, whether the number of pixels in which a luminance value is equal to or greater than a predetermined value, is equal to or greater than a predetermined number, in the image of the changed cutout area.

If halation is included (S4: YES), the control portion 21 judges whether the setting is such that halation is hidden, that is, whether the setting is such that the halation region is not shown (S5). The judgement of S5 is made based on a setting by the user.

For example, when the insertion portion 5 is inserted, the insertion operation is performed aiming at the lumen, so the user may find the insertion operation easier with halation set to be hidden. If halation is set to be hidden (S5: YES), the control portion 21 judges the halation region, and determines a correction amount to correct the cutout area so that the halation region will not be included in the image of the cutout area (S6).

Also, the control portion 21 corrects the cutout area, based on the determined correction amount (S7). That is, the process of S6 is a process that corrects the cutout area so that the halation region will not be included in the cutout area that was changed in S3.

After S6, the control portion 21 executes cutout processing (S8). That is, the control portion 21 executes processing that cuts out the front view image and the side view image to be displayed on the display portion 4 from the region OR indicated by the dotted line in FIG. 3, i.e., the region OR of the object image formed on the image pickup surface of the image pickup device 14a, based on the cutout area corrected in S7.

Then, the control portion 21 executes exposure control such that the cutout image comes to be at the proper exposure (S9).

When no halation is included (S4: NO) and when halation is not hidden (S5: NO), the process proceeds on to S8, where the control portion 21 cuts out the cutout area changed in S3 from the region OR of the object image formed on the image pickup surface of the image pickup device 14a.

The cutout processing will now be described in detail with reference to FIG. 5.

From the state indicated by G1 in FIG. 5, it shall be assumed that the user has performed a bending operation to bend the bending portion 5b to the right direction.

When the bending operation is performed and the bending portion 5b bends a certain amount in a bending direction MR indicated by the alternate long and two short dashes arrow in FIG. 5, i.e., to the right direction, the control portion 21 changes the cutout area CA to be cut out from the object image taken on the image pickup surface of the image pickup device 14a, based on the bending direction and bending angle of the distal end portion 5a according to the bending direction MR and the bending amount. That is, in the endoscope image displayed on the display portion 4, the cutout area CA is changed so as to include a region that is not shown, which is a region to the right side in the direction of the bending by the user, in the image taken on the image pickup surface of the image pickup device 14a. As a result, the user is able to view more of the region in the direction of the bending, i.e., to the right side where the user wishes to observe, than can be viewed according to the bending amount.

In other words, when the user performs a bending operation, the cutout area CA is changed as shown in G2 of FIG. 5, so as to include an image of a region that is taken in the image pickup device 14a but is not shown, which is a region in the bending operation direction, and the changed cutout area CA is displayed on the display portion 4. As a result, the user is able to more quickly observe the image in the bending operation direction that is the direction in which the user wishes to see.

Note that, as described above, as one setting, the user is able to set whether to display a halation region, in the endoscope system 1 from the setting input portion 25. If the user does not wish to have an endoscope image that includes halation displayed, due to a bending operation, the user sets the halation region to be hidden.

When the kind of setting is made, the cutout area CA is corrected so as to not include the halation region.

For example, as shown in FIG. 5, in S3, the cutout area CA is changed, as shown in G2, for example, based on the bending direction and bending angle of the distal end portion 5a. In FIG. 5, the cutout area CA is changed so as to move by a movement amount d1 to the right direction as shown in G2, by the process of S3.

However, a halation region HA is set so as to be hidden, so if a halation region such as the halation region HA indicated by the alternate long and two short dashes line is in the cutout area CA that has been changed by the movement amount d1, the cutout area CA is corrected so as not to include the halation region HA (S7). The control portion 21 can, in the case of FIG. 5, make the width in the left-right direction of the halation region HA a correction amount d2, for example. In FIG. 5, the cutout area CA that has been moved by the movement amount d1 determined in S3 is corrected to the cutout area CA that has been moved to the left by the correction amount d2, as shown in G3. As a result, in FIG. 5, the cutout area CA is changed from cutout state G1 to G3 and displayed on the display portion 4.

That is, if a halation region such as the halation region HA indicated by the alternate long and two short dashes line is in the image of the cutout area changed by S3, the cutout area CA is corrected so that the halation region HA will not be displayed on the display portion 4 (S7).

Therefore, when the image signal of the side view image changed in S3 includes a predetermined pixel region that is a halation pixel region, the process of S7 corrects the amount of change in the image signal of the side view image so that the side view image to be displayed on the display portion 4 does not include the halation region.

Note that the movement amount d1 is determined linearly or incrementally (i.e., nonlinearly), according to the direction in which the distal end portion of the distal end portion 5a faces and the change amount in the direction, or the operation direction and the bending operation amount of a bending operation. The movement amount d1 according to the change amount and the like may be able to be set by the user.

Furthermore, whether the movement amount d1 is determined linearly, or is determined incrementally, according to the bending operation amount and the like of the bending operation, may be set by the user.

If the user is able to set the change amount of the direction in which the distal end portion of the distal end portion 5a faces according to the bending angle and the like, and whether the change amount is changed linearly or incrementally, an endoscope image according to the bending operation and the like that is desired by the user is able to be displayed.

The example above is an example in which the bending operation is to the right direction, but when bending is to the left, up, or down direction, the cutout area CA is changed so as to include more of the image in the bending direction. Alternatively, the same applies with any combination of directions of up, down, left, and right, and the cutout area CA is changed so as to include more of the image in the combination of directions.

Therefore, as described above, according to the embodiment, an endoscope system that enables quick observation when the direction of field of view of the endoscope having a wide angle of field of view is changed, is able to be provided.

In the embodiment and another embodiment that will be described later, the first image of the object from the front, which is the first direction, (the first object image, the front view image) is defined as a primary image that is an image to be primarily displayed, because the image is needed to be observed almost constantly when operating the endoscope system 1.

Also, the second image of the object from the side, which is the second direction, (the second object image, the side view image) is defined as a secondary image, because the image may not be necessary to always be primarily displayed in comparison with the primary image.

Note that, based on the definitions of the primary image and the secondary image described above, when disposing a simple observation window that faces forward in order to improve insertability forward, which is the insertion axis direction, in a side-view type endoscope in which the primary observation window is always facing to the side of the insertion portion 5, for example, the side view image may be defined as the primary image and the front view image may be defined as the secondary image, and processing based on the first embodiment described above may be performed.

That is, the region (in the first direction) that obtains the primary image may be one of a region that includes the direction in front of the insertion portion that is substantially parallel to the longitudinal direction of the insertion portion or a region that includes the direction to the side of the insertion portion that is substantially orthogonal to the longitudinal direction of the insertion portion, and the region (in the second direction) that obtains the secondary image may be the other of the region that includes the direction in front of the insertion portion or the region that includes the direction to the side of the insertion portion.

Next, modifications of the embodiment will be described.

### (First Modification)

In the example described above, the cutout area CA is changed so as to include an image of the region that is not displayed, in the bending direction, but the cutout area CA may also be enlarged to include even more of the image in the region that is not displayed, in a direction orthogonal to the cutout direction.

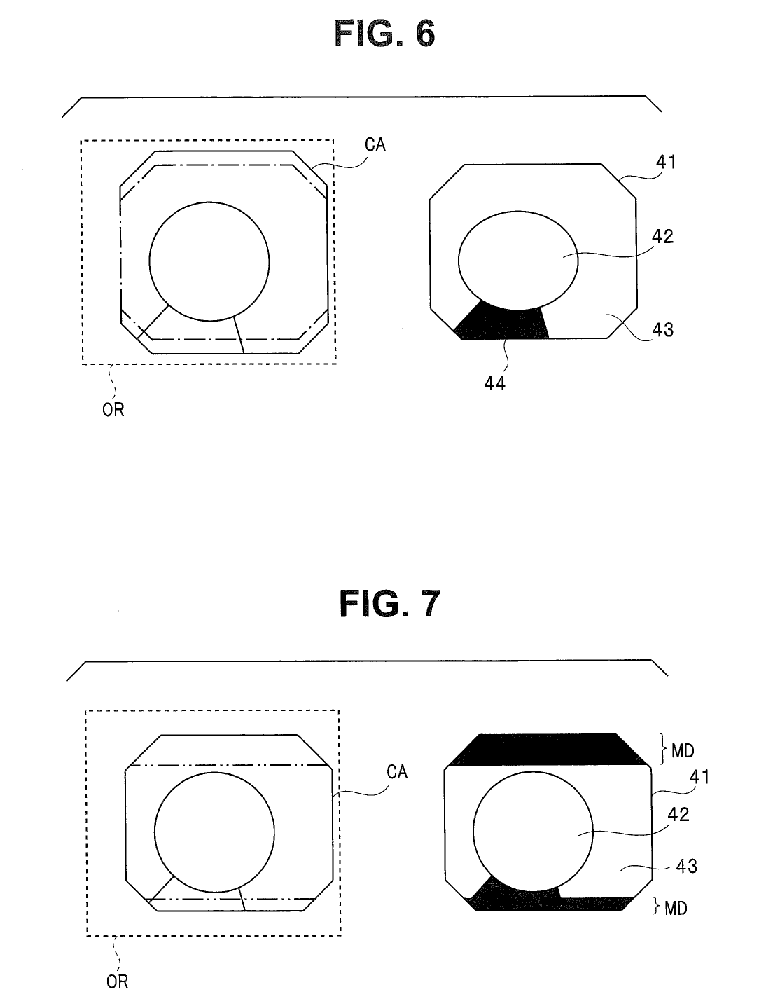

FIG. 6 is a view of a cutout area CA and the display image 41 of the display portion 4, according to the first modification.

In the modification, when the bending operation to the right direction described above is performed, the control portion 21 moves the cutout area CA to the right direction, and enlarges the cutout area CA in the vertical direction as indicated by the solid line from the area indicated by the alternate long and short dash line in FIG. 6.

That is, when a change in the direction in which the distal end portion of the insertion portion 5 faces is detected, the image signal of the side view image in the direction orthogonal to the direction of the change is changed in S3 so as to include both an image in the direction of the change, and an image of the region that is not displayed of the side view image.

The display image 41 of the display portion 4 is reduced in the vertical direction linearly or incrementally according to the bending operation amount, so as to become an image that is compressed in the vertical direction by the amount that the display image 41 was enlarged in the vertical direction.

In the first modification as well, the cutout area CA is changed in the bending direction of the user, so the user is able to quickly observe the region that he/she wishes to see. Also, the display area in the direction orthogonal to the direction in which the user wishes to see is also enlarged, so the user is able to also quickly observe the surrounding region in the direction in which he/she wishes to see.

The example above is an example in which a bending operation is performed to the right direction, but when bending is to the left, up, or down direction, the cutout area CA is changed such that the image is enlarged in the direction orthogonal to the bending direction.

Note that whether the cutout area CA is to be enlarged in the direction orthogonal to the bending direction, as in the first modification, may be performed by setting by the user.

Moreover, note that the enlargement amount or enlargement factor of the cutout area CA in the direction orthogonal to the bending direction may also be set by the user.

### (Second Modification)

In the example described above, the cutout area CA is changed such that the image in the bending direction is included, but in addition, the direction orthogonal to the cutout direction may be prevented from being displayed.

FIG. 7 is a view of the cutout area CA and the display image 41 of the display portion 4, according to a second modification.

In the modification, when the bending operation to the right direction described above is performed, the control portion 21 moves the cutout area CA to the right direction, and masks, linearly or incrementally, according to the bending operation amount, a predetermined area in the vertical direction of the cutout area CA indicated by the alternate long and two short dashes line, such that the vertical direction of the cutout area CA is not displayed.

That is, when a change in the direction in which the distal end portion of the insertion portion 5 faces is detected, the image signal of the side view image in the direction orthogonal to the direction of the change is changed in S3 so as to include the image in the direction of the change, and not display a portion of the side view image.

As a result, the display image 41 of the display portion 4 has a region MD that is masked and thus not displayed in the vertical direction.

In the second modification as well, the cutout area CA is changed in the bending direction of the user. Therefore, the user is able to quickly observe the region he/she wishes to see, and a portion of the region in the direction orthogonal to the direction in which the user wishes to see will not be displayed, so the user is able to quickly observe, watching only the image that he/she wishes to see.

The example above is an example in which a bending operation is performed to the right direction, but when bending is performed to the left, up, or down direction, the image in a direction orthogonal to the bending direction is masked.

Note that, whether a portion in the direction orthogonal to the bending direction is to be masked and not displayed, as in the second modification, may be performed by setting by the user.

Moreover, note that the area that is prevented from being displayed in the direction orthogonal to the bending direction may also be set by the user according to the change amount in the direction in which the distal end portion of the distal end portion 5a faces.

### (Third Modification)

In the example described above, the cutout area CA is changed so as to include the image in the bending direction, but in addition, when the changed cutout area CA includes halation, whether to make the halation region the proper exposure in the exposure control in S9 may be set.

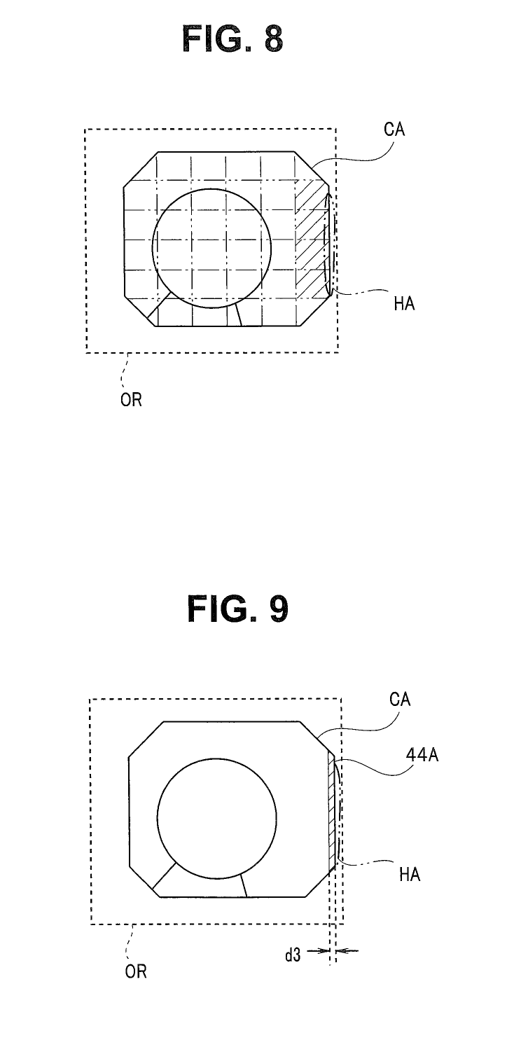

FIG. 8 is a view of a plurality of division regions DA for an exposure determination in the cutout area CA, according to the third modification.

The cutout area CA is divided into a plurality (36 in FIG. 8) of division regions DA beforehand, as indicated by the alternate long and two short dashes line in FIG. 8. That is, the cutout area CA cut out from the image taken on the image pickup surface of the image pickup device 14a is divided into the plurality of division regions DA.

When the cutout area CA includes halation, a judgement is made as to which of the division regions DA, among the plurality of division regions DA, the halation region HA is in.

For example, with FIG. 8, the control portion 21 is able to judge that the halation region HA is in four division regions DA on the right side in the cutout area CA.

For example, when the user sets the halation region HA to be displayed at the proper exposure in order to confirm an affected area or the like when pulling out the insertion portion 5, the entire endoscope image becomes darker, but the control portion 21 performs exposure control based on the luminance value of the four regions that include the halation region, in S9. That is, the four regions that include the halation region are set as photometric regions. The exposure control is performed by controlling the light amount of illumination light from the illumination control portion 24, for example.

Also, when the user sets the regions other than the halation region HA to be displayed at the proper exposure, the entire endoscope image becomes lighter, but the control portion 21 performs the exposure control based on the luminance value of the regions other than the four regions. That is, the regions other than the four regions that include the halation region are set as photometric regions.

That is, S9 is an exposure control portion that performs exposure control of the side view image, and performs exposure control based on the luminance of the halation region, which is a predetermined pixel region, or the luminance of the regions other than the halation region.

Furthermore, when halation is included, the proper exposure value used in the exposure control may be changed.

### (Fourth Modification)

In the example above, the cutout area CA is changed so as to include the image in the bending direction, but in addition, when halation is included in the cutout area CA that has been changed, masking may be performed so that the halation region will not be displayed.

FIG. 9 is a view of the cutout area CA and the display image 41 of the display portion 4, according to the fourth modification.

In the modification, instead of correcting the cutout area as shown in G3 of FIG. 5, the halation region HA is prevented from being displayed by a mask 44A (shown by the diagonal lines) having a width d3 in the horizontal direction so that the halation region HA is not displayed. The region of the mask 44A is darkened like the mask 44, on the display portion 4.

That is, when a change in the direction in which the distal end portion of the insertion portion 5 faces is detected, and the image of the cutout area after the change includes a halation region, the image signal of the side view image is changed in S3 so as to mask so that the halation region will not be displayed.

### Second Embodiment

One image pickup device is built into the distal end portion 5a of the insertion portion 5 of the endoscope of the first embodiment in order to obtain an object image from at least two directions. However, two or more image pickup devices are built into the distal end portion 5a of the insertion portion 5 of the endoscope of the embodiment in order to obtain an object image from at least two directions.

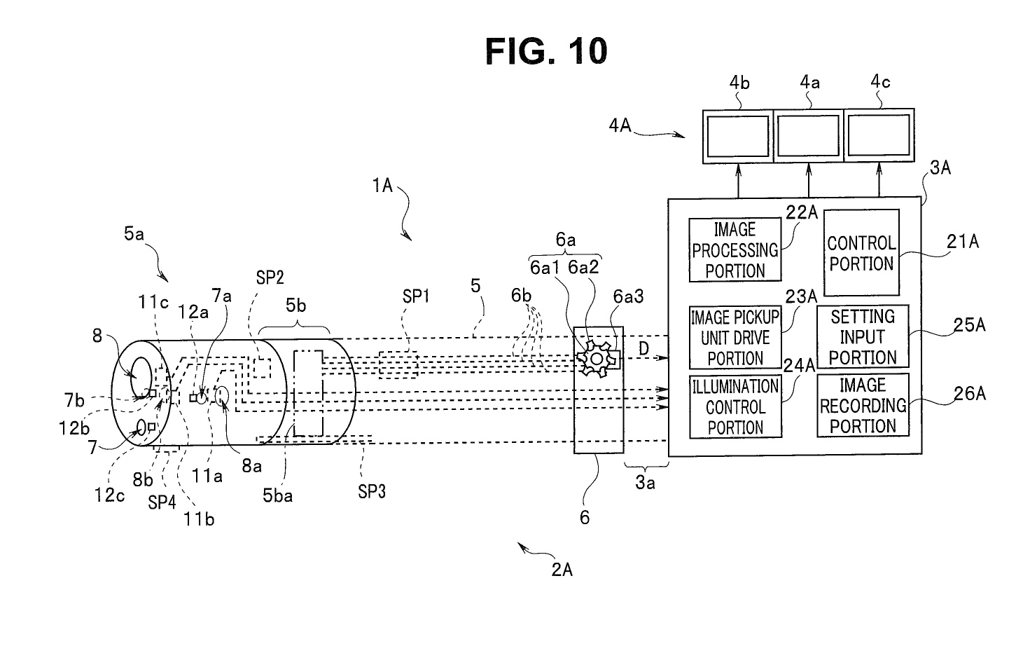

FIG. 10 is a configuration diagram showing the configuration of an endoscope system according to the embodiment. An endoscope system 1A of the embodiment has substantially the same configuration as the endoscope system 1 of the first embodiment, so components that are the same as components of the endoscope system 1 will be designated by the same reference characters, and descriptions of the components will be omitted. Only the configuration which is different will be described.

The illumination window 7 and the observation window 8 for a front view, and the two illumination windows 7a, 7b and two observation windows 8a, 8b for a side view, are provided on the distal end portion 5a of the insertion portion 5 of the endoscope 2.

That is, the endoscope 2 has the two illumination windows 7a and 7b in addition to the illumination window 7, and has the two observation windows 8a and 8b in addition to the observation window 8. The illumination window 7a and the observation window 8a are for a first side view, and the illumination window 7b and the observation window 8b are for a second side view. Also, the plurality, here two, of observation windows 8a and 8b are disposed at substantially equal angles in the circumferential direction of the insertion portion 5.

The distal end portion 5a of the insertion portion 5 has a distal end rigid member, not shown. The illumination window 7 is provided on a distal end surface of the distal end rigid member 61, and the illumination windows 7a and 7b are provided on an outer peripheral surface of the distal end rigid member 61 such that illumination light beams are emitted in directions opposite to each other.

An image pickup unit 11a for the first side view is arranged inside the distal end portion 5a, behind the observation window 8a, and an image pickup unit 11b for the second side view is arranged inside the distal end portion 5a, behind the observation window 8b. An image pickup unit 11c for the front view is arranged behind the observation window 8 for the front view.

Each of the three image pickup units 11a, 11b, 11c that are image pickup portions has an image pickup device, is electrically connected to a processor 3A, is controlled by the processor 3A, and outputs an image pickup signal to the processor 3A. Each of the image pickup units 11a, 11b, 11c is an image pickup portion that photoelectrically converts an object image.

Accordingly, the observation window 8 is disposed facing the direction in which the insertion portion 5 is inserted, in the distal end portion 5a of the insertion portion 5, and the observation windows 8a and 8b are disposed facing the outer radial direction of the insertion portion 5, on side surface portions of the insertion portion 5.

That is, the observation window 8 configures a first image obtaining portion that is provided on the insertion portion 5 and obtains an image of a first object image from the front, which is a first direction, and each of the observation windows 8a and 8b configures a second image obtaining portion that is provided on the insertion portion 5 and obtains a second image (second object image) from a second region that is a region that includes a side direction that differs from the front.

In other words, the first image from the first region is an object image in the first direction that includes a direction in front of the insertion portion 5 that is substantially parallel to the longitudinal direction of the insertion portion 5, and the second image from the second region is an object image in the second direction that includes a direction to the side of the insertion portion 5 that is substantially orthogonal to the longitudinal direction of the insertion portion 5.

The image pickup unit 11c is an image pickup portion that photoelectrically converts the image from the observation window 8, and the image pickup units 11a and 11b are image pickup portions that photoelectrically convert the two images from the observation windows 8a and 8b, respectively.

An illuminating light emitting device 12a for the first side view is arranged inside the distal end portion 5a, behind the illumination window 7a, and an illuminating light emitting device 12b for the second side view is arranged inside the distal end portion 5a, behind the illumination window 7b. An illuminating light emitting device 12c for the front view is arranged behind the illumination window 7 for the front view. The illuminating light emitting devices (hereinafter, referred to as light emitting devices) 12a, 12b, 12c are light emitting diodes (LEDs), for example.

Accordingly, the illumination window 7 corresponding to the light emitting device 12c is an illumination portion that emits illumination light toward the front, and the illumination windows 7a and 7b corresponding to the light emitting devices 12a and 12b, respectively, are illumination portions that emit illumination light toward the sides.

The processor 3A has a control portion 21A, an image processing portion 22A, an image pickup unit drive portion 23A, an illumination control portion 24A, a setting input portion 25A, and an image recording portion 26A.

The control portion 21A has the same function as the control portion 21 described above, and includes a central processing unit (CPU), and ROM, RAM and the like, and controls an overall endoscope apparatus.

The image processing portion 22A has the same function as the image processing portion 22 described above, and generates an image signal based on the image pickup signals from the three image pickup units 11a, 11b, 11c, and outputs the image signal to a display portion 4A, under the control of the control portion 21A.

In particular, the image processing portion 22A has the same function as the image processing portion 22 described above, and generates an image, cuts out the image, changes the cutout area, and enlarges or reduces the cutout image, and the like, under the control of the control portion 21A.

The image pickup unit drive portion 23A has the same function as the image pickup unit drive portion 23 described above, and drives the three image pickup units 11a, 11b, 11c. Each of the driven image pickup units 11a, 11b, 11c generates an image pickup signal, and supplies the image pickup signal to the image processing portion 22A.

The illumination control portion 24A is a circuit that controls the on/off and light amount of the light emitting devices 12a, 12b, 12c.

The setting input portion 25A and the image recording portion 26A also have the same functions as the setting input portion 25 and the image recording portion 26 described above, respectively.

The display portion 4A has three display apparatuses 4a, 4b, 4c. Each of the display apparatuses 4a, 4b, 4c receives, from the processor 3A, a signal of an image to be displayed that has been converted to a display signal. The front view image is displayed on the screen of the display apparatus 4a, the first side view image is displayed on the screen of the display apparatus 4b, and the second side view image is displayed on the screen of the display apparatus 4c.

That is, two side view images exist, and the image processing portion 22A outputs an image signal of the front view image and image signals of the two side view images to the display portion 4A, such that the front view image is arranged in the center on the display portion 4A and the two side view images sandwich the front view image.

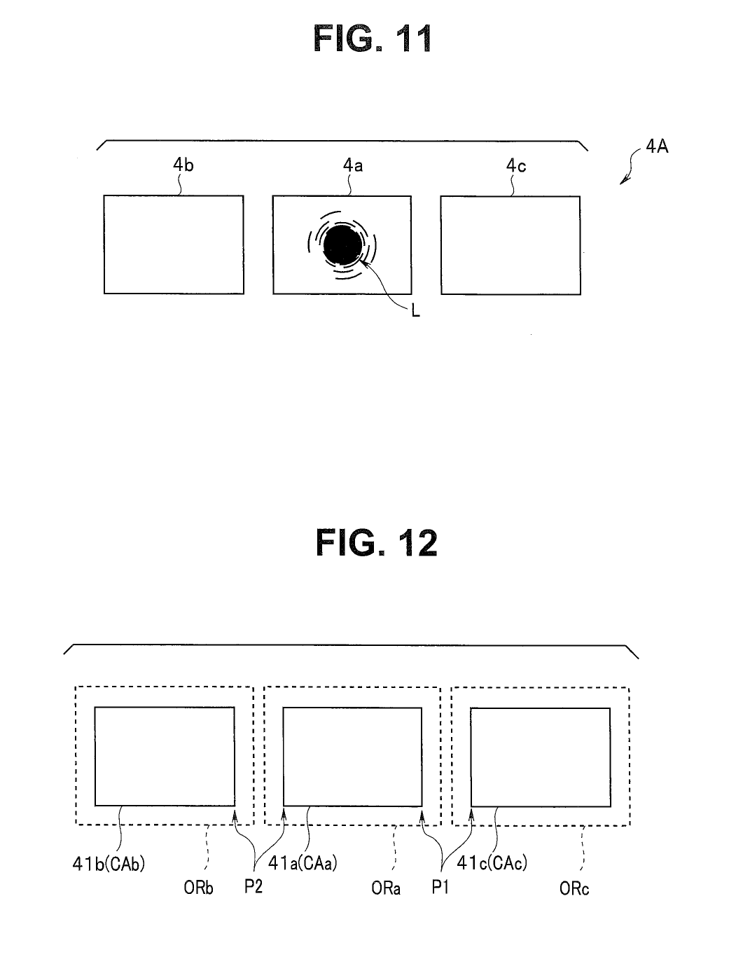

FIG. 11 is a view showing an example of a display screen of an endoscope image displayed on a display portion 4A. FIG. 11 shows the state in which the three display apparatuses 4a, 4b, 4c of the display portion 4A are disposed.

The front view image is displayed on the display apparatus 4a, the first side view image is displayed on the display apparatus 4b, and the second side view image is displayed on the display apparatus 4c. In FIG. 11, an image when the user is performing an examination by inserting the insertion portion into the large intestine is displayed. A lumen L is displayed in the front view image. The two side view images are displayed on both sides of the front view image, so a wide angle endoscope image is displayed on the display portion 4A.

FIG. 12 is a view illustrating an example of a display screen of an endoscope image displayed on the display portion 4A, and an object image region of the three image pickup units 11a, 11b, 11c.

Display images 41a, 41b, 41c, which are endoscope images displayed on the screens of the respective display apparatuses 4a, 4b, 4c of the display portion 4A are rectangular images.

The display image 41a displayed on the display apparatus 4a in the center is generated from an obtained image obtained by the image pickup unit 11c. The display image 41b displayed on the display apparatus 4b on the left side is generated from an obtained image obtained by the image pickup unit 11a. The display image 41c displayed on the display apparatus 4c on the right side is generated from an obtained image obtained by the image pickup unit 11b.

The respective display images 41a, 41b, 41c are generated by cutting out images in cutout areas CAa, CAb, CAc corresponding to the respective display images, in regions ORa, ORb, ORc, which are indicated by the dotted lines in FIG. 12, respectively. Each of the regions ORa, ORb, ORc represents an area of the object image taken formed on the image pickup surface of the corresponding image pickup device.

In order to guarantee the continuity of the three images displayed on the display portion 4A, positions P1, P2 of a boundary of two adjacent cutout areas are adjusted and set such that the left end of the cutout area CAc of the region ORc, and the right end of the cutout area CAa of the region ORa are in the same position in the object image, and the left end of the cutout area CAa of the region ORa, and the right end of the cutout area CAb of the region ORb are in the same position in the object image.

### (Operation)

The control portion 21A of the embodiment performs the processing shown in FIG. 4 that is described in the first embodiment. However, in the embodiment, a change in the cutout area according to the bending operation is made with respect to the respective regions ORa, ORb, ORc of the three image pickup units 11a, 11b, 11c.

In FIG. 4, when the cutout area is changed according to the bending operation (S3), if the bending direction is to the right direction, for example, the user wishes to see an image in the bending direction, so the cutout area of the side view image in the bending direction is changed.

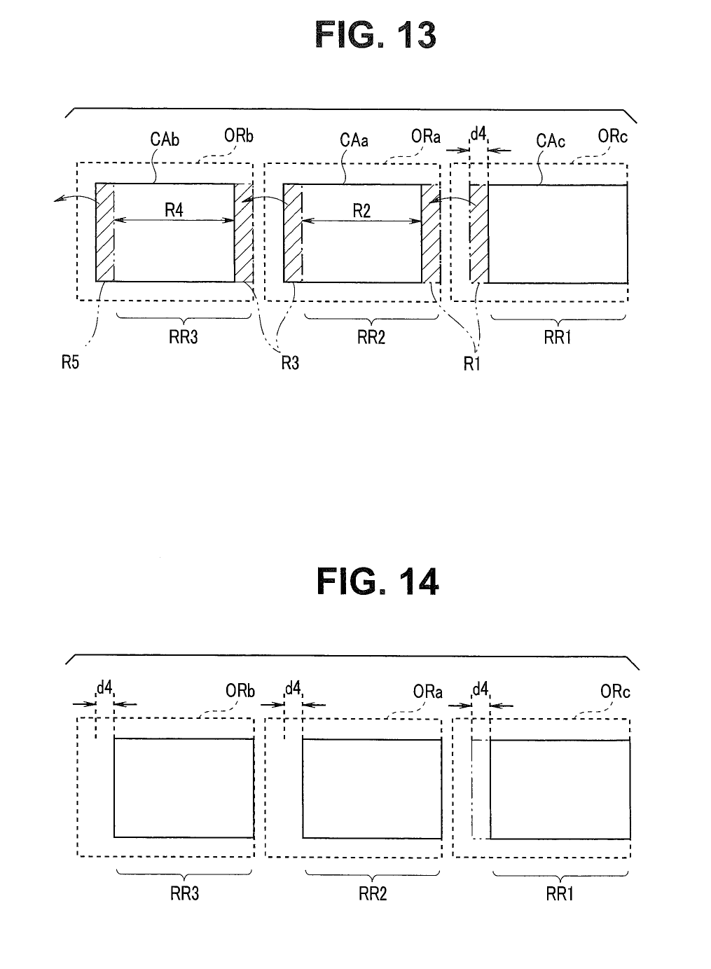

FIG. 13 is a view illustrating a change in the cutout area when cutting out a region of an image displayed on the display portion 4A from an object image taken on each image pickup surface of the three image pickup units 11a, 11b, 11c, according to the embodiment.

When a bending operation to the right side is performed, the cutout area CAc from the region ORc of the endoscope image generated by the image pickup unit 11b that generates the second side view image is changed according to the change amount in the bending direction and the direction in which the distal end portion of the insertion portion 5 faces.

In FIG. 13, the cutout area CAc in the region ORc that indicates the area of the object image formed on the image pickup surface of the image pickup unit 11b moves to the right side by an amount d4 according to the change amount in the direction in which the distal end portion of the insertion portion 5 faces. As a result, more of the image in the direction in which the user wishes to see is displayed on the display portion 4A.

Moreover, at the time, the control portion 21A may not only change the cutout area of the region ORc, but may also perform substitution processing on portions of the images in the cutout areas CAb and CAc in the respective regions ORb and ORc of the other image pickup units 11a and 11c.

As shown in FIG. 13, the cutout area CAc in the region ORc is moved to the right direction by the amount d4, so the image displayed on the display apparatus 4c is the area indicated by RR1 in FIG. 13.

Therefore, the control portion 21A displays an image obtained by combining a region R1 that is the amount d4 on the left side of the cutout area CAc before the cutout area CAc is moved, with a region R2 on the right side of the cutout area CAa of the region ORa, on the display apparatus 4a.

The region on the right side of the cutout area CAa that is combined with the region R1 is a region that excludes the amount d4 on the left side of the cutout area CAa. Therefore, the image displayed on the display apparatus 4a becomes the area indicated by RR2 in FIG. 13.

Furthermore, the control portion 21A displays an image obtained by combining a region R3 of the amount d4 on the left side of the cutout area CAa before the cutout area CAa is moved, with a region R4 on the right side of the cutout area CAb of the region ORb, on the display apparatus 4b. The region on the right side of the cutout area CAb that is combined with the region R3 is a region that excludes the amount d4 on the left side of the cutout area CAb. Therefore, the image displayed on the display apparatus 4b becomes the area indicated by RR3 in FIG. 13.

The image of a region R5 of the amount d4 on the left side of the cutout area CAb is not used for display.

Note that the endoscope image displayed on the display apparatus 4b may not be combined with the image of the region R3, and the image of the region R4 may be enlarged in the horizontal direction.

Furthermore, note that in the example described above, the image in the region not displayed is made to move. However, the cutout areas may also be changed according to the change amount in the direction in which the distal end portion of the insertion portion 5 faces.

FIG. 14 is a view showing a state in which the cutout area of each of the regions ORa, ORb, ORc is made to move in the bending direction according to the bending operation amount.

As shown in FIG. 14, the cutout area CAc of the region ORc is changed by the amount d4 in the bending direction, and the cutout areas CAa and CAb of the regions ORa and ORb are also changed by the amount d4 in the bending direction. A method such as the method illustrated in FIG. 14 also yields results similar to the results obtained by the method illustrated in FIG. 13.

In the embodiment as well, the correction of the cutout area (S7) depending on whether halation is included, in FIG. 4, may also be made according to the setting.

Furthermore, in the embodiment as well, whether to perform the exposure control (S9) in FIG. 4 such that the halation region becomes the proper exposure may also be determined according to the setting.

Therefore, as described above, according to the embodiment, an endoscope system that enables quick observation when changing a direction of field of view of an endoscope having a wide angle of field of view is able to be provided.

Next, modifications of the second embodiment will be described.

### (First Modification)

The first modification of the first embodiment may also be applied to the embodiment as well. That is, the cutout area in a direction orthogonal to the bending direction, in an image in the bending direction, may also be enlarged according to the setting, and the user may set the enlargement amount or enlargement factor of the cutout area CA in the direction orthogonal to the bending direction.

### (Second Modification)

The second modification of the first embodiment may also be applied to the embodiment as well. That is, the image in a direction orthogonal to the cutout direction may be masked so as not to be displayed, according to the setting, and the user may set the area that is not to be displayed, which is in the direction orthogonal to the bending direction.

### (Third Modification)

The third modification of the first embodiment may also be applied to the embodiment as well. That is, when halation is included in the cutout area CA that has been changed, whether to make the halation region the proper exposure in the exposure control in S9 may also be set.

### (Fourth Modification)

The fourth modification of the first embodiment may also be applied to the embodiment as well. That is, when halation is included in the cutout area that has been changed, masking may be performed so that the halation region is not displayed.

### (Fifth Modification)

In the second embodiment described above, when the bending portion 5b is bent in either the left or right direction, the side view image of the direction opposite the bending direction will be displayed, but the side view image may also be hidden.

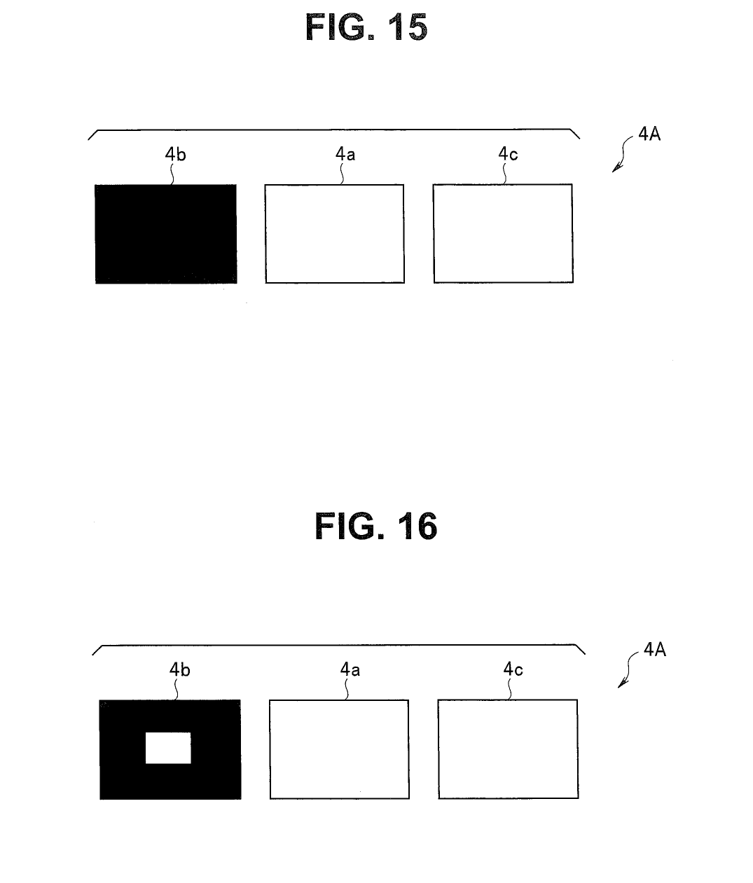

FIG. 15 is a view illustrating a display state of the display portion 4A when bending is performed to the right side, according to a fifth modification. When the bending direction is to the right side, the second side view image that is of the right side is displayed on the display apparatus 4c, but the first side view image that is of the opposite direction is not displayed on the display apparatus 4b. This is because the user wishes to view the bending direction, so the image of the opposite direction does not have to be displayed.

Note that, with FIG. 15, the endoscope image of the display apparatus 4b may also gradually become darker from the right side toward the left side, instead of the display apparatus 4b not displaying the image at all.

Furthermore, note that when the bending direction is to the right side, the size of the first side view image on the left side, which is in the direction opposite the right side, may be made smaller.

FIG. 16 is a view illustrating another example of a display state of the display portion 4A when bending is performed toward the right side, according to the fifth modification. As shown in FIG. 16, the size of the first side view image on the left side, which is in the direction opposite the right side, is small on the display apparatus 4b.

### (Sixth Modification)

In the second embodiment and the modifications described above, the mechanism for realizing the functions of illuminating and observing to the side, together with the mechanism for realizing the function of illuminating and observing to the front, is built into the insertion portion 5. However, the mechanism for realizing the function of illuminating and observing to the side may also be separate and detachable from the insertion portion 5.

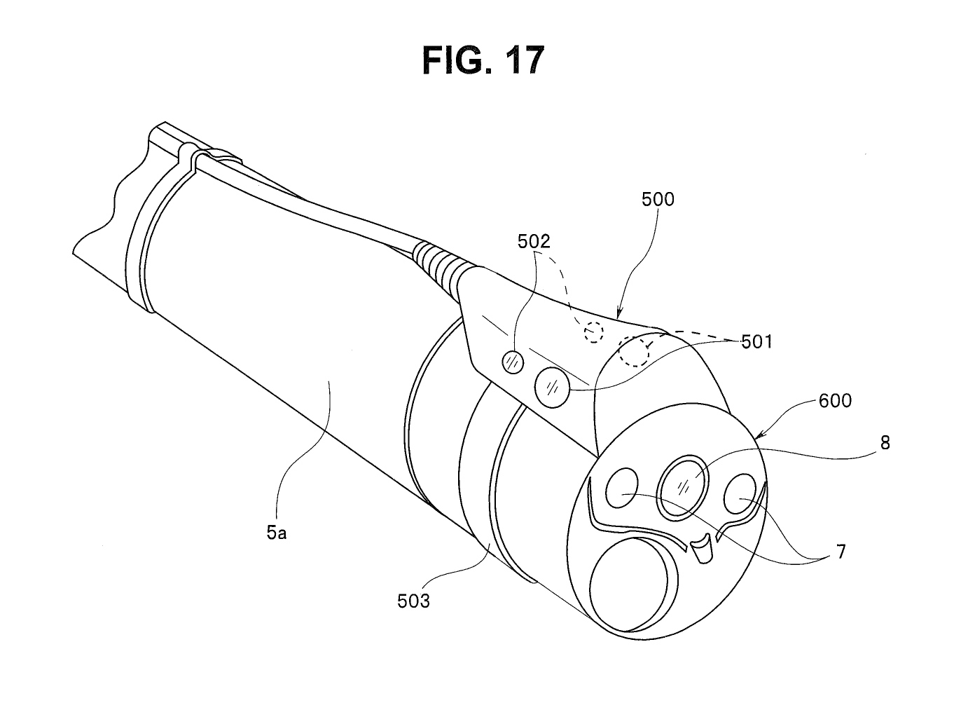

FIG. 17 is a perspective view of the distal end portion 5a of the insertion portion 5 to which a unit for side observation is attached, according to a sixth modification. The distal end portion 5a of the insertion portion 5 has a front viewing unit 600. A side viewing unit 500 has a configuration in which the side viewing unit 500 can be detached from the front viewing unit 600 by a clip portion 503.

The side viewing unit 500 has two observation windows 501 for obtaining images in the left and right directions, and two illumination windows 502 for illuminating in the left and right directions.

The processor 3A and the like turns on and off the respective illumination windows 502 of the side viewing unit 500 in accordance with a frame rate of the front view, and can obtain and display an observation image such as described in the embodiments described above.

As described above, according to the respective embodiments and the respective modifications described above, it is possible to provide an endoscope system that enables quick observation when changing the direction of field of view of an endoscope having a wide angle of field of view.

The present invention is not limited to the embodiments described above. Various modifications and improvements and the like are possible without departing from the scope of the present invention.

What is claimed is:

1. An endoscope system comprising:
an insertion portion configured to be inserted into a subject;
an objective optical system provided on a distal end of the insertion portion and configured to form an optical image of an object inside the subject;
an image pickup portion configured to pick up the optical image;
a bending portion configured to cause the distal end of the insertion portion to bend;
a bending direction detection portion configured to detect a bending direction of the distal end of the insertion portion by the bending portion;
an image processing portion configured to perform cutout processing on an image picked up by the image pickup portion, such that an area of field of view expands in the bending direction detected by the bending direction detection portion; and
an exposure control portion configured to perform exposure control based on a brightness of the image cut out by the image processing portion.

2. The endoscope system according to claim 1, further comprising:
an operation member configured to be capable of operating bending of the bending portion, wherein
the bending direction detection portion detects the bending direction by detecting an operation applied to the operation member.

3. The endoscope system according to claim 1, wherein when bending of the distal end of the insertion portion is not detected by the bending direction detection portion, the image processing portion cuts out a predetermined region in an image picked up by the image pickup portion, as the cutout processing, and when bending of the distal end of the insertion portion is detected by the bending direction detection portion, the image processing portion moves a cutout region such that a field of view expands in the detected bending direction from the predetermined region, as the cutout processing.

4. The endoscope system according to claim 3, wherein when a halation region is included in the cutout region changed according to a detection result of the bending direction detection portion, the image processing portion moves the cutout region further so that the halation region is not cut out.

5. The endoscope system according to claim 3, wherein when a halation region is included in the cutout region changed according to a detection result of the bending direction detection portion, the image processing portion performs masking so that the halation region is not displayed.

6. The endoscope system according to claim 3, wherein the exposure control portion is capable of switching between and setting a mode that performs exposure control such that a halation region included in the cutout region changed according to a detection result of the bending direction detection portion becomes a proper exposure, and a mode that performs exposure control such that a region in the cutout region, other than the halation region, becomes a proper exposure.

7. The endoscope system according to claim 3, wherein the image processing portion performs processing to move the cutout region so that the field of view expands in the detected bending direction from the predetermined region according to a detection result of the bending direction detection portion, enlarges the cutout region in a direction orthogonal to the bending direction, and converts an aspect ratio of an image cut out according to a region which is the region that is moved and enlarged, to an aspect ratio of the region before the region is enlarged.

8. The endoscope system according to claim 3, wherein the image processing portion moves the cutout region so that the field of view expands in the detected bending direction from the predetermined region according to a detection result of the bending direction detection portion, and masks an image portion of an end portion in a direction orthogonal to the bending direction such that the image portion is not displayed.

9. The endoscope system according to claim 1, wherein
the objective optical system includes
a first objective optical system configured to form an optical image of the object in a front view region that includes an insertion portion front direction that is substantially parallel to a longitudinal direction of the insertion portion, on a predetermined region of an image pickup surface of the image pickup portion, and
a second objective optical system configured to form an optical image of the object in an insertion portion side region that is substantially orthogonal to the longitudinal direction of the insertion portion, on a region that is different from the predetermined region of the image pickup surface.

10. The endoscope system according to claim 1, wherein
the objective optical system includes
a first objective optical system configured to form an optical image of the object in a front view region that includes an insertion portion front direction that is substantially parallel to a longitudinal direction of the insertion portion, and
a second objective optical system configured to form an optical image of the object in an insertion portion side region that is substantially orthogonal to the longitudinal direction of the insertion portion; and
the image pickup portion includes
a first image pickup portion configured to pick up the optical image of the object in the front view region that is formed by the first objective optical system, and
a second image pickup portion configured to pick up the optical image of the object in the side region that is formed by the second objective optical system.

11. The endoscope system according to claim 10, wherein the image processing portion performs the cutout processing on an image picked up by the first image pickup portion, such that an area of field of view is expanded in a bending direction detected by the bending direction detection portion, with respect to the image picked up by the first image pickup portion.

12. The endoscope system according to claim 11, wherein the image processing portion further performs processing to reduce an information amount of an image taken by picking up an image of the side region on a side opposite the bending direction detected by the bending direction detection portion.

13. The endoscope system according to claim 12, wherein the processing to reduce the information amount, performed by the image processing portion, is processing to not display an image taken by picking up the image of the side region on the side opposite the bending direction.

14. The endoscope system according to claim 12, wherein the processing to reduce the information amount, performed by the image processing portion, is processing to reduce an image taken by picking up the image of the side region on the side opposite the bending direction.
